(19) United States
(12) Plant Patent Application Publication
(10) Pub. No.: US 2002/0133860 P1
(43) Pub. Date: Sep. 19, 2002

(54) Kalanchoe plant named 'Pippijn'

(75) Inventor: Andres Smaal, (US)

Correspondence Address: GARDNER, CARTON & DOUGLAS, Suite 3400, 321 North Clark Street, Chicago, IL, 60610, US

(73) Assignee: A.B. BREEDING BV

(21) Appl. No.: 10/024,685

(22) Filed: Dec. 14, 2001

(30) Foreign Application Priority Data
Apr. 7, 2000 (EP) 20000415
Jan. 11, 2001 (CA) 01-2522

(51) Int. Cl. A01H 5/00

(52) U.S. Cl. PLT/341

(57) ABSTRACT

A new and distinct cultivar of Kalanchoe plant named Pippijn is provided. This new cultivar is characterized by its bright red flowers, dark green foliage, compact growth habit and excellent basal branching.

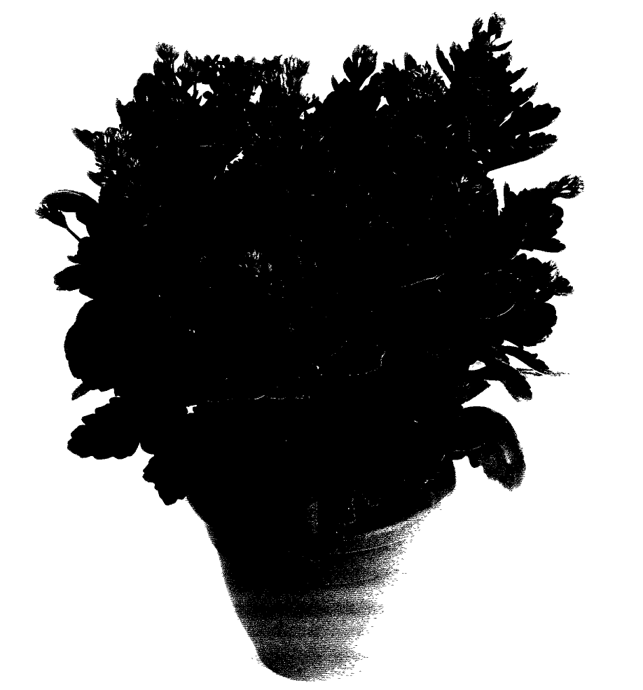

### BACKGROUND OF THE INVENTION

The present invention comprises a new and distinct Kalanchoe plant, botanically known as Kalanchoe blossfeldiana, and hereinafter referred to by the cultivar name ‘Pippijn’.

The new cultivar was developed by the inventor through a controlled breeding program during August 1995 in The Netherlands. The female (seed) parent of ‘Pippijn’ was the proprietary Kalanchoe selection designated 93114-057 (not patented) characterized by its red flowers. The male (pollen) parent of ‘Pippijn’ was the proprietary Kalanchoe selection designated 94077 (not patented) characterized by its purple flowers and dark green foliage. ‘Pippijn’ was discovered and selected as a flowering plant within the progeny of the stated cross by the inventor in June 1996.

Asexual reproduction of the new cultivar by terminal cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits bright red flowers,

(b) forms dark green foliage,

(c) exhibits a good basal branching character and

(d) exhibits a compact upright growth habit.

The new cultivar of the present invention can be compared to ‘Calbuco’ (USPPAF). In side by side comparisons with ‘Calbuco’, it is found that the new cultivar is less compact, more floriferous and exhibits smaller flowers of a darker red color. A detailed comparison can be found in Table A.

### BRIEF DESCRIPTION OF PHOTOGRAPH

The accompanying photograph shows as nearly true as it is reasonably possible to make the same in color illustrations of this type, typical flower and foliage characteristics of the new cultivar. The plants were grown in a greenhouse at West Chicago, Ill., U.S.A.

### DETAILED BOTANICAL DESCRIPTION

The cultivar Pippijn has not been observed under all possible environmental conditions to date. Accordingly, it is possible that the phenotype may vary somewhat with variations in the environment, such as temperature, light intensity, and day length.

The chart used in the identification of colors described herein is the R.H.S. Colour Chart of The Royal Horticultural Society, London, England. The color values were determined on Jan. 4, 2001 between 9:00 and 9:30 am under natural daylight. The plants used for the following observations and measurements were produced from cuttings taken from stock plants and grown in West Chicago, Ill. in 10 cm pots for sixteen (16) weeks. Plants were grown in a double poly carbonate covered greenhouse under conditions comparable to those used in commercial practice while utilizing a soilless growth medium and maintaining temperatures of approximately 82° F. during the day and approximately 66° F. during the night. Plants were exposed to three weeks of long days followed by 9 weeks of short days. Growth regulators were applied as follows: B nine, 3000 ppm at days 17 and 28 and 1500 ppm day 42.

Classification:

Botanical.—Kalanchoe blossfeldiana, cultivar Pippijn.

Parantage:

Male (pollen) parent.—Proprietary Kalanchoe blossfeldiana selection #94077.

Female (seed) parent,—Proprietary Kalanchoe blossfeldiana selection #93114-057.

Propagation:

Type cutting,—Terminal tip.

Time to Initiate Roots,—Approximately 7-14 days with the shorter times generally being experienced in the summer and the longer times in the winter.

Time to develop roots,—Approximately 21 days.

Root Description,—Fibrous, branching.

Plant description:

Habit of Growth,—Moderately vigorous with good basal branching. A mature plant, at 16 weeks after the planting of an unrooted cutting, commonly measures approximately 13.1 cm in height and approximately 11.9 cm in width.

Branching habit,—Typically forms one main stem 11 cm in length and 5 mm in diameter and 11 lateral branches approximately 6 cm in length and 3 mm in diameter.

Plant Form,—Upright and mounded.

Foliage,—Form: Simple. Arrangement: Opposite. Shape: Ovate with obtuse apex, cuneate base, and crenate margin. Texture: Glabrous, succulent. Size of mature foliage: Approximately 6.7 cm in length and approximately 4.9 cm in width. Color of mature foliage: Upper surface between 137A and 139A; lower surface closest to 137B. Petiole length: Approximately 1.3 cm. Petiole color: Closest to 137A.

Flower description:

Flowering Habit,—Freely flowering.

Natural Flowering Season,—Early January. Flowering time is dependent on temperature, light intensity and other growing conditions.

Flowering response time,—Approximately 11 weeks.

Inflorescence type,—Dichasial cymes.

Flowers Borne,—Above foliage.

Flower longevity,—Approximately 7 days.

Fragrance,—None.

Quantity of Flowers,—Approximately 24 flowers on main stem and 15 flowers on each lateral branch.

Flower buds,—Shape: Elliptical, becoming elongated as it matures. Size just before opening: Length is approximately 1.1 cm, width is approximately 5 mm. Color: 43D.

Flower,—Salverform. Corolla: Diameter is approximately 1 cm, depth is approximately 3 mm and aspect is cupped. Corolla tube length is approximately 9.5 mm and diameter is approximately 3 mm.

Petals,—Number: Four. Shape: Ovate with cuspidate apex and rounded base. Approximate size: 7 mm in length, 6 mm in width. Aspect: Slightly convex. Texture: Glabrous. Appearance: Somewhat iridescent.

Mature Flower Color.—Upper surface of petals is closest to 53B fading to slightly more blue than 53B at senescence; lower surface of petals is closest to 43D.

Sepals,—Number: Four. Shape: Lanceolate with acute apex, entire margin. Size: 6 mm in length amd 2 mm in width. Texture: Glabrous. Color: Upper and lower surface: 144B.

Peduncle,—Length: Approximately .9 cm in length and 2 mm in diameter.

Pedicel length,—Approximately 5 mm.

Reproductive Organs,—Androecium: 8 stamens arranged in two groups of four,—one group being shorter than the other. Anther shape is oblong and flat, length is <1 mm. Pollen color is 6C. Gynoecium: Stigma shape is flat, appearance is crystalline, colorless and transparent, size is <1 mm in length. Style length is approximately 2 mm, color is 151 D. Ovary is 4 celled, 6.5 mm in length. Color is 144B.

Seed development: Seed development has not been observed.

Disease resistance: Resistance to known Kalanchoe pathogens has not been observed.

TABLE A

| CHARACTERISTIC | ‘PIPPIJN’ | ‘CALBUCO’ |
|---|---|---|
| PLANT HEIGHT | 13.1 cm | 9.5 cm |
| NUMBER OF BRANCHES | 11 | 13 |
| FLOWER DIAMETER | 1 cm | 1.8 cm |
| PETAL COLOR-FULLY OPENED- | 53B | Between 54A and 51A |
| UPPER SURFACE | | |
| APPROXIMATE NUMBER OF | 186 | 50 |
| FLOWERS PER PLANT | | |
| COROLLA DIAMETER | .9 cm | 1.8 cm |

I claim:

1. A new and distinct cultivar of Kalanchoe plant named ‘Pippijn’ substantially as herein shown and described, which:
(a) exhibits dark red flowers,
(b) forms dark green foliage,
(c) exhibits a good basal branching character and
(d) exhibits a compact upright growth habit.
